(19) United States
(12) Patent Application Publication
(10) Pub. No.: US 2010/0267142 A1
(43) Pub. Date: Oct. 21, 2010

(54) SCALABLE PACKED-BED CELL CULTURE DEVICE

(76) Inventors: Gary Wang, Taichung (TW); King-Ming Chang, Taichung (TW); Lewis Ho, Lawrenceville, GA (US)

Correspondence Address: Muncy, Geissler, Olds & Lowe, PLLC, 4000 Legato Road, Suite 310, FAIRFAX, VA, 22033, US

(21) Appl. No.: 12/761,810

(22) Filed: Apr. 16, 2010

Related U.S. Application Data
(60) Provisional application No. 61/202,901, filed on Apr. 16, 2009.

(51) Int. Cl. C12N 5/071; C12M 1/36; C12N 1/20

(52) U.S. Cl. 435/395; 435/286.2; 435/252.1

(57) ABSTRACT

A scalable packed-bed cell culture device includes a matrix vessel, a mixing vessel, a communicating means, a driving means and a controlling means. The matrix vessel includes porous matrixes packed therein. The mixing vessel includes a mixing means configured for mixing a culture medium. The communicating means is connected between the matrix vessel and the mixing vessel. The driving means is configured for driving the culture medium to flow between the matrix vessel and the mixing vessel. The controlling means configured for controlling the culture medium to submerge the porous matrixes at high level, and to emerge the porous matrixes at low level. An inoculation method and a culture method for scalable packed-bed cell culture device is also herein provided for eliminating the limitation of aeration or oxygenation during culture, alleviating the gradient effect, eliminating the channeling effect in conventional packed-bed bioreactors.

### BACKGROUND OF THE INVENTION

1. Field of the Invention

The present invention relates to a scalable packed-bed cell culture device, and more particularly to a scalable packed-bed cell culture device, an inoculation method and a cell culture method.

2. Description of the Prior Art

Large-scale cell culture processes have been developed extensively over years for the growth of bacteria, yeast and molds, all of which typically possess robust cell walls and/or extra cellular materials thus, are more resilient. The structural resilience of these microbial cells is a key factor contributing to the rapid development of highly-efficient cell culture processes for these types of cells. For example, bacterial cells can be grown in very large volumes of liquid medium using vigorous agitation, culture stirring and gas sparging techniques to achieve good aeration during growth while maintaining viable cultures. In contrast, the techniques to culture cells such as eukaryotic cells, animal cells, mammalian cells and/or tissue are more difficult and complex because these cells are far more delicate and fragile than microbial cells. These cells can be easily damaged by excessive shear forces, resulted from vigorous aeration and agitation required for microbial cultures in conventional bioreactors.

A general example of a cell-cultivating system is roller bottles. Each roller bottle can provide an area of only 850-3000 cm² for cultivating cells. Therefore, thousands of roller bottles are simultaneously taken care of in the factories, requiring a great deal of labor. Automation of the roller-bottle cell-cultivating system can save labor, but is expensive.

Another example of cell-cultivating systems is a stir tank. The tank has microcarriers inside for growing cells thereon. In this example, however, stirring culture medium and gassing cells considerably threaten growth of the cells. Furthermore, the operation conditions need to be changed when the dimensions of the stirring tank are enlarged. Changes of the operation conditions greatly delay the product development.

Another example of cell-cultivating systems is hollow fibers, by which the cell density can be up to 10⁸/ml. In this example, however, the reactor for cultivating cells is a plug-flow type. When the cell density increases to a predetermined level, the cells at the rear end of the reactor cannot obtain enough nutrition and the growth will be inhibited. To avoid such a situation, the reactor generally is not made large, which is the major disadvantage of the hollow fiber reactor.

Packed-bed bioreactor contains porous matrixes for cell growth and protects cells from shear. Due to the high surface area provided by porous matrixes, cell density can be higher than the other systems. Usually a density of 5˜10×10⁷ cells/ml matrix can be easily achieved. However, most of the packed-bed bioreactors are one directional recirculation flow along with the packed-bed forming a so-called plug flow pattern. Due to the plug flow pattern, both nutrient and oxygen are depleted along with the flow path and form gradient that limits the scale-up capability in the system. We call this a “gradient effect”. The gradient effect occurs in all plug flow design devices such as hollow fiber bioreactors and packed-bed bioreactors that all have scale-up limitation.

Besides, the flow pattern along with the packed-bed cross section is not homogeneous. Medium flow quickly and smoothly through those regions in low packing density with higher permeability, and flow slowly or cease flow in those regions with higher packing density with lower permeability. This is so called channeling effect. The channeling effect impedes cell growth and causes cell death in those regions with high packing density.

Conventional inoculation method is to submerge the matrix vessel with culture medium and then introduce concentrated inoculums with high density into the vessel. A driving means drive the inoculums flow through the packed-bed in one direction. Due to the packed-bed functions as a depth filter, inoculums are trapped from top of the packed-bed to the bottom and cause a gradient distribution of the cells in the packed-bed during early inoculation phase. These problems are usually tempted to be alleviated by increasing the flow rate of the culture medium to reduce the gradient effect and heterogeneous distribution of cells. However, higher flow rate poses shear stress to the cells, and a pressure drop along with the bed height also limits the flow rate.

Due to the gradient effect, the channeling effect and the heterogeneous distribution of the cells during inoculation phase, the scale of the packed-bed bioreactor is greatly limited. Generally, the scale of a packed-bed type bioreactor is limited within 10 to 30 liters. While at least 10 fold increases will be essential to make it useful for industrial manufacture purpose. (“Packed-bed bioreactors for mammalian cell culture: Bioprocess and biomedical applications, F. Meuwly et. al. Biotechnology Advances Vol. 25, Issue 1, January-February 2007, Pages 45-56).

The scale limitation thus becomes a major bottle neck of a packed-bed type bioreactor. Therefore, eliminating the nutrient/oxygen gradient, the channeling effect, and improving the inoculation distribution under reasonable flow rate is the key to unlock the scale limitation of a packed-bed bioreactor. Traditional design of packed-bed culture device, such as U.S. Pat. No. 5,501,971, issued to Freedman et al., entitled “Method and apparatus for anchorage and suspension cell culture”, discloses a method and apparatus for cultivating cells in a reactor that includes a basket-type packed bed and an internal liquid cell growth medium recirculation device consisting of a stirrer. The traditional design will have all above mentioned drawback such as gradient, channeling effect and gradient distribution of cells that limits the bed scale below 10 L.

Others have tried to overcome the scale-up problem in packed-bed system. For example, U.S. Pat. No. 5,766,949, issued to Liau et al. (“Liau”), entitled “Method and Apparatus for Cultivating Anchorage Dependent Monolayer Cells” describes a cell-cultivating system in which the culture medium oscillates up and down with respect to a growth substrate in an attempt to improve the oxygenation of the cells. Liau, however, presents many disadvantages. One disadvantage of this system is the complexity of Liau's apparatus. The Liau system requires two external storage tanks and a separate growth chamber which holds a series of vertical substrate plates. Multiple peristaltic pumps are required to circulate the growth medium from one storage tank through the culture chamber and then into another storage tank and then back to the first storage tank. Introduction of contaminants is very likely given the complexity and the reliance of the components for the Liau apparatus which are external to the culture chamber, for example, the external tubings, storage tanks, and pumps. Further, sterilization is difficult and laborious due to a relatively large amount of components to the apparatus and the size of apparatus. Another problem presented by Liau is that the flow of the culture medium through the system would create hydrodynamic shear forces that can easily disrupt and dislodge cells from the substrate plates, thus, reducing the viability of the cells. Furthermore, the vertical substrate plates also discourage cell adhesion since cells that cannot adhere immediately to the plates will simply fall and accumulate at the bottom of the plates and, eventually, most of these cells die. Thus, the culture has a reduced viability, the protein production decreases correspondingly and the system would require continual restarting which is highly inefficient and counterproductive. Moreover, due to the complexity of the system, the harvesting of any secreted protein or cellular product would be cumbersome and time consuming. Lastly, when the growth medium is lowered with respect to the growth substrate plates, the cells become exposed to air, i.e., gaseous environment directly, and thus, may result in cell death.

U.S. Pat. No. 6,323,022, issued to Chang et al., entitled “Highly efficient cell-cultivating device” describes a cell-cultivating system includes a plurality of culture tanks and a driving device. The culture tanks communicate with each other and have culture medium inside. The driving device forces the culture medium to flow between the culture tanks so as to vertically oscillate medium levels in the culture tanks. The major disadvantages presented by Chang are that the packed-bed completely relied on the self-forming static mixer to work when the medium flowing through. This will cause several problems such as settlement of cells before adhering on the culture matrixes, heterogeneous nutrient conditions that might affect the cell growth, difficult to adjust pH by adding alkali solution, difficult to measure pH, and dissolve oxygen in the culture medium due to lack of mixing in the culture tanks. Limited oxygen supply due to the lack of mixing and sparging in the medium tanks poses another problem in this invention.

U.S. Pat. No. 7,033,823 B2, issued to King-Ming Chang, entitled “Cell-cultivating device” describes a device and method for cell cultivation. The device consists of a hollow cylinder in which a porous, fibrous matrix is located between an upper and a lower basket, the matrix serving as bedding for the cells. An upper chamber is situated above, and a lower chamber below the bedding matrix. The lower chamber essentially consists of a compressible bellows-type bag, by means of which liquid cell growth medium can be recirculated to the upper chamber. The major disadvantages presented by Chang is the lower chamber has little mixing capability and causes cell settlement problems during inoculation phase. Due to no mixing, it is difficult to measure pH and adjust pH in the culture chamber. Besides, the compressible bellows-type bag design poses threaten of leakage that limits the system to be scaled up.

Given the importance of cell and tissue culture technology in biotechnology research, pharmaceutical research, academic research, biopharmaceutical manufacturing and in view of the deficiencies, obstacles and limitations exist in the prior art described the present invention overcomes the obstacle and remedies the deficiencies in the prior art by teaching and disclosing a method and an apparatus for cell and tissue culturing that fulfills the long-felt need for a novel method and apparatus to culture cells and tissues that is more reliable, less complex, more efficient, less cumbersome, capable of increasing production scale and producing a higher yield of cellular by-products generated from the cells.

### RELATED APPLICATIONS/PATENTS & INCORPORATION BY REFERENCE

This application claims priority to provisional application Ser. No. 61/202,901 filed Apr. 16, 2009 entitled “Scalable fixed-bed culture device” incorporated herein by reference, together with any documents therein cited and any documents cited or referenced in their cited documents.

### SUMMARY OF THE INVENTION

The present invention is directed to a scalable packed-bed cell culture device, an inoculation method and a cell culture method that enable scaling up and achieving high cell density and high yield.

The present invention is also directed to a scalable packed-bed cell culture device, an inoculation method and a culture method that could eliminate the limitation of aeration or oxygenation during culture, can alleviate the gradient effect, eliminate the channeling effect in conventional packed-bed bioreactors.

In one embodiment, the present invention provides a scalable packed-bed cell culture device includes a matrix vessel, a mixing vessel, a communicating means, a driving means and a controlling means. The matrix vessel includes porous matrixes packed therein. The mixing vessel includes a mixing means configured for mixing a culture medium. The communicating means is connected between the matrix vessel and the mixing vessel. The driving means is configured for driving the culture medium to flow between the matrix vessel and the mixing vessel. The controlling means configured for controlling the culture medium to submerge the porous matrixes at high level, and to emerge the porous matrixes at low level.

In another embodiment, the present invention provides an inoculation method for a scalable packed-bed cell culture including providing a matrix vessel comprising porous matrixes packed therein, wherein a plurality of void space is formed among the porous matrixes; and introducing an inoculum medium having an inoculum into the matrix vessel, wherein the inoculum medium flows through the void space and submerges the void space, whereby the inoculum is distributed onto the surface of the porous matrixes. The major difference between conventional inoculation method and the novel inoculation method is during inoculation, the matrix vessel is filled with culture medium in the conventional method, and a concentrated inoculums are introduced from top of the vessel filled with culture medium. While the novel method is started with a matrix vessel without submerging with culture medium, and a homogeneous inoculum solution is introduced into the matrix vessel.

In yet another embodiment, the present invention provides a cell culture method comprising providing the scalable packed-bed cell culture device; introducing an inoculum medium having an inoculum into the matrix vessel, wherein the inoculum medium flows through the void space and submerges the void space, whereby the inoculum is distributed onto the surface of the porous matrixes; and dual-directional flowing of the culture medium and oxygenation between the matrix vessel and the mixing vessel.

Other advantages of the present invention will become apparent from the following descriptions taken in conjunction with the accompanying drawings wherein are set forth, by way of illustration and example, certain embodiments of the present invention.

### BRIEF DESCRIPTION OF THE DRAWINGS

The following description, given by way of example, is not intended to limit the invention to any specific embodiments described. The description may be understood in conjunction with the accompanying Figures, incorporated herein by reference.

FIGS. 1 to 4 are side views illustrating the cell-cultivating apparatus according to preferred embodiments of the present invention.

FIGS. 5a to 5e are schematic diagrams illustrating the cell inoculation method and devices thereof.

FIG. 6 is a histogram illustrating cell distribution result using the inoculation method of the present invention.

FIG. 7 is a histogram illustrating cell distribution result with conventional inoculation method.

FIG. 8 is a broken line graph illustrating the glucose consumption using the scalable cell culture device of the present invention.

FIG. 9 is a line graph illustrating the pH profile using the scalable cell culture device of the present invention.

FIG. 10 is a line graph illustrating the dissolved oxygen profile using the scalable cell culture device of the present invention.

FIG. 11 is a scatter diagram illustrating cell distribution in the 10 L packed-bed vessel sampling before virus infection in the vertical direction from top to the bottom.

FIG. 12 is a broken line graph illustrating virus production profile using the cell culture device.

### DESCRIPTION OF THE PREFERRED EMBODIMENT

The following detailed description, given by way of example, is not intended to limit the invention to any specific embodiments described. The detailed description may be understood in conjunction with the accompanying figures, incorporated herein by reference. Without wishing to unnecessarily limit the foregoing, the following shall discuss the invention with respect to certain preferred embodiments.

The embodiments of the present invention can be used to culture any cells, such as eukaryotic and prokaryotic cells, particularly animal cells and/or mammalian cells. The embodiments of the present invention can be used to produce any products generated from cells, such as recombinant protein, enzyme and/or viruses.

In a preferred embodiment of the present invention, the cell-cultivating device contains two chambers: a mixing vessel and a matrix vessel.

The mixing vessel comprises a plurality of openings for introducing or removing culture medium or for other purposes. The openings for air inlet and outlet contain an air filter. A mixing means is installed inside or outside the chamber. For the mixing means inside the mixing vessel, a propeller, a stir blade is preferred; for outside the mixing vessel, a shaker, rocker is preferred. The mixing vessel is preferably flexible and disposable, and of course it could be a rigid metal, glass or plastic container as well. A sparger is installed inside the mixing vessel optionally to provide additional oxygenation capability. The sparger increases the dissolved oxygen in the mixing vessel by either surface aeration through agitation or/and additionally sparging with air bubbles. The matrix vessel may also be emerged the air or oxygen intermittently for increasing the dissolved oxygen in the matrix vessel. At least a tube is used for communicating the mixing vessel and the matrix vessel. The mixing vessel is supported in a platform that could provide temperature control and mixing to homogenize the culture medium inside the mixing vessel. The mixing vessel can also be installed with pH, DO or temperature probe for monitoring and process control.

The matrix vessel has openings for air inlet and outlet, openings on the top of the chamber for introducing cells, culture medium or buffer solution. The openings for air inlet and outlet contain an air filter. Porous matrixes are disposed inside the matrix vessel. The matrix vessel and/or mixing vessel may be supported on a platform with a driving means which could move the matrix vessel and/or matrix vessel up and down vertically so as to adjust the relative altitude between the matrix vessel and the mixing vessel. The matrix vessel can also be held on a stationary platform if other driving means such as air compressor, air pump or pressure/vacuum pump is used to drive the medium flow. The matrix vessel is temperature controlled by an external means.

The porous matrixes in the matrix vessel form a loosely packed matrix that can function as a depth filter to capture cells during culture medium movement in order to maximize cell entrapment, anchored and/or embedded. The porous matrixes also maximize air-medium contact by providing a thin air-medium interface when the porous matrixes emerge from the growth medium. The porous matrixes are a porous substrate of any size and shape, e.g. plate, pebble, or stripe, and can be constructed from any configuration. The porous matrixes are randomly disposed inside the chamber and forming a loosely packed depth filter. Each of the porous matrixes works as a small filter to entrap cells or substrates for cell attachment. However, the void space between each of the porous matrixes is large enough to avoid clogging or fouling by the cells during the medium flow process. Porous matrixes may include may include woven carriers, non-woven carriers, plates, porous carriers made of ceramics, porous carriers made of polymer or tissue engineering scaffolds. More specifically, the porous matrixes are preferably non-woven fibrous matrix which can entrap the cells as a filter. More specifically, the porous matrixes are a macroporous with pore size from 50 um to 200 um and with porosity larger than 70%. More specifically, the porous matrixes according to the preferred embodiment of invention provide a maximum amount of surface area for cell entrapping, adhesion, growth, and oxygenation. The system in accordance with the preferred embodiment of invention also provides an easy way to collect culture medium containing cellular products with less burden of losing cells due to that most cells are entrapped by the porous matrixes. The cell-cultivating apparatus of the preferred embodiment of invention also protects cells from being directly exposed to any air, gas bubbles or any shear forces generated by an influx of gas, thus, avoiding any detrimental effects to the cells.

Reference is now made to the figures by way of examples and they are by no way limiting the scope of the present invention.

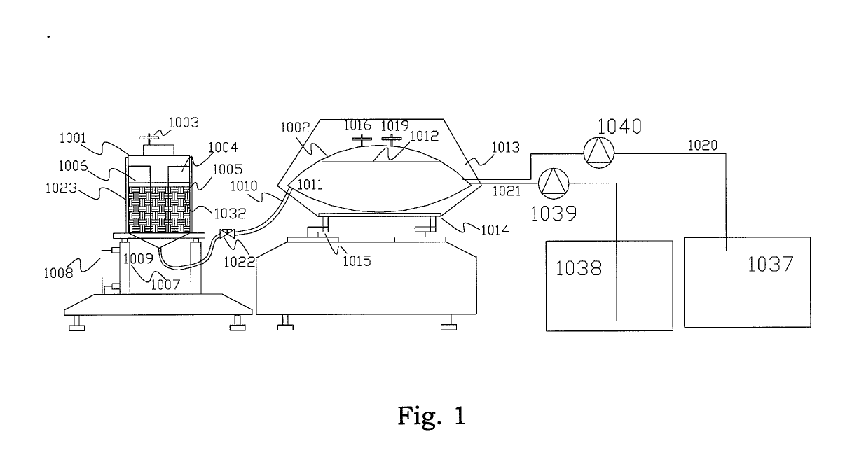

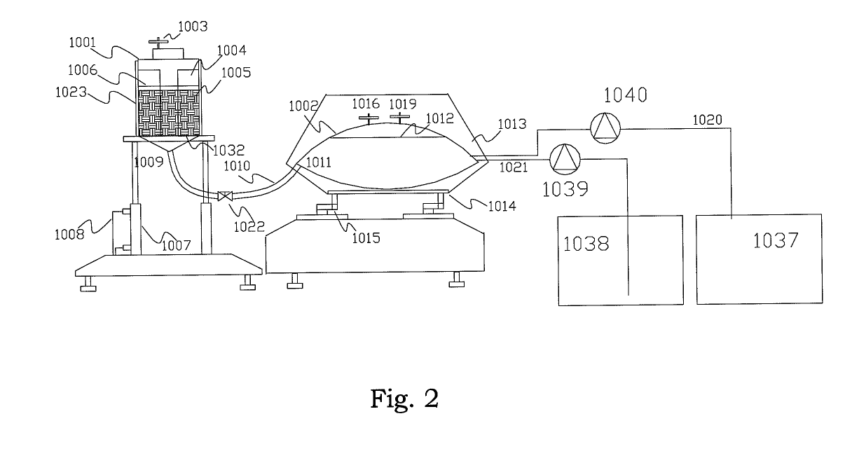

Referring to FIG. 1 and FIG. 2, a side view of the cell-cultivating apparatus of one of the preferred embodiment of the present invention is presented. Reference is made to the figures. A matrix vessel 1001 contains an opening with air filter 1003, and an opening 1009 at the bottom of the matrix vessel 1001 connected with a pipe or tube 1010 to the mixing vessel 1002. An on/off valve 1022 is set on the pipe 1010 to control the flow between the matrix vessel 1001 and the mixing vessel 1002. A porous matrix means 1005 is filled in the matrix vessel 1001. The matrix vessel 1001 is secured on the holder 1023 surrounded by a heating pad 1004 and is mounted on the driving means 1007. The driving means 1007 in the figure is an oil or air cylinder, two level sensors 1008 are mounted on the cylinder to control the upper limit and the lower limit of the movement of the cylinder. The mixing vessel 1002 is a flexible bag with one opening 1011 and is connected by a pipe or tube 1010 to the matrix vessel 1001. Two openings connected with air filters 1016, 1019 for air in and out and for medium aeration during mixing. A tube 1021 is connected between the mixing vessel 1002 and a reservoir or feed container 1038. A peristaltic pump 1039 is mounted in the tube 1021 between the reservoir 1038 and the mixing vessel 1002 to transfer the culture medium into the mixing vessel 1002. Another tube 1020 is connected to a reservoir or harvest container 1037. A peristaltic pump 1040 is mounted in the tube 1020 between the reservoir 1037 and the mixing vessel 1002 to harvest the culture medium from the mixing vessel 1002. The mixing vessel 1002 is secured on a platform with a container 1013 and a heating pad 1014 to provide proper temperature environment, a shaker 1015 that can rotate orbitally or vibrate the container 1013 to mix the culture medium inside the mixing vessel 1002. An inoculating device is configured for introducing an inoculating medium into the porous matrixes.

In FIG. 1, the matrix vessel 1001 is at the low limit level relative to the liquid level 1012 in the mixing vessel 1002, so that the culture medium could flow from the mixing vessel 1002 to the matrix vessel 1001 through the tube 1010 and submerge the porous matrix means 1005 in the matrix vessel 1001 and raise the liquid level 1006 to the upper limit in the matrix vessel 1001.

Referring to FIG. 2, the cell-cultivating apparatus of one of the preferred embodiment of invention is the same as FIG. 1 except the matrix vessel 1001 is at the high limit level relative to the liquid level 1012 in the mixing vessel 1002, so that the culture medium could flow from the matrix vessel 1001 to the mixing vessel 1002 through the tube 1010 and expose the porous matrix means 1005 in the matrix vessel 1001.

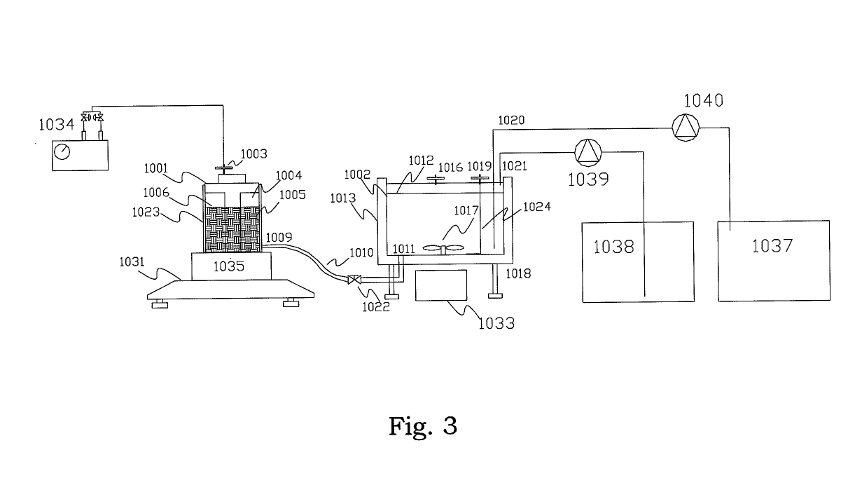

Referring to FIG. 3 the cell-cultivating apparatus of one of the preferred embodiment of invention, the mixing vessel 1002 has a propeller 1017 built inside the chamber. The matrix vessel 1001 is in stationary and the driving means for the culture medium flow between the mixing and matrix vessel is a pneumatic means 1034, which is an air/vacuum pump with solenoid valves and timer control set on a tube connected to air filter 1003 to control the flow of culture medium between the mixing vessel 1002 and the matrix vessel 1001 by pressure and vacuum. The medium level in the matrix vessel 1001 is further controlled by a load cell 1035 which can be a level sensor as well. The mixing vessel 1002 contains a magnetic stir blade or a magnetic bar 1017 inside the chamber and is driven by a magnetic stirrer 1033 outside of the mixing vessel 1002.

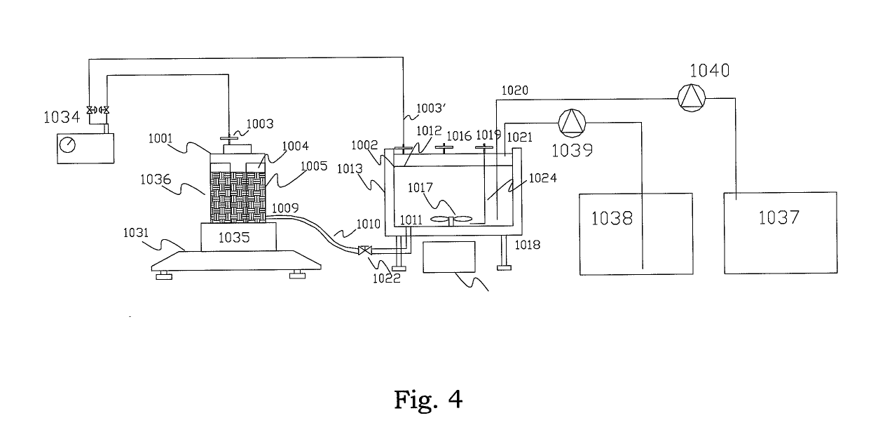

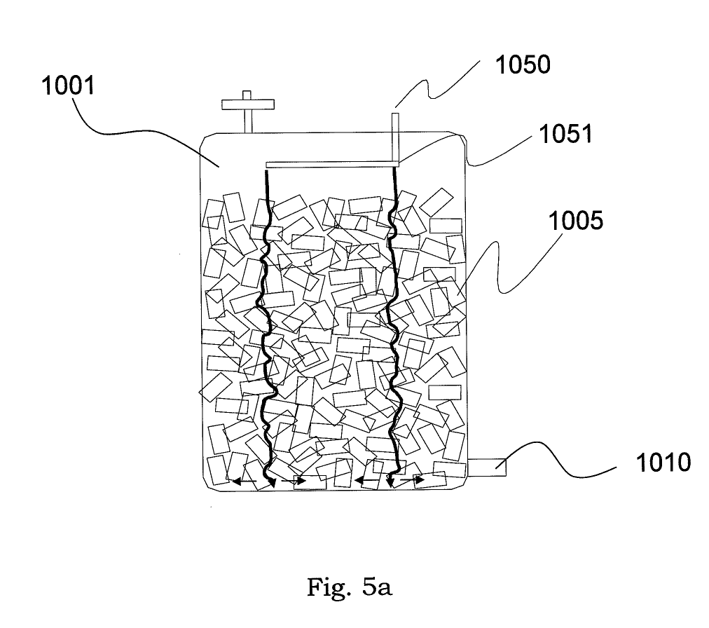

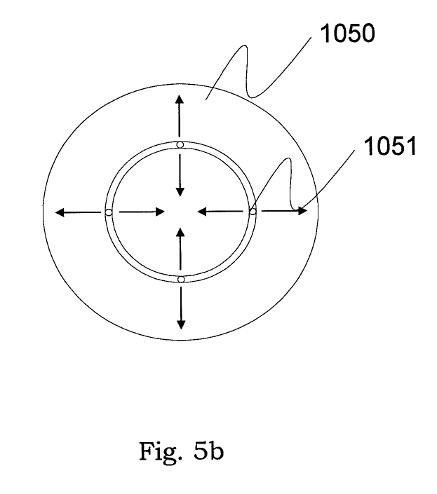

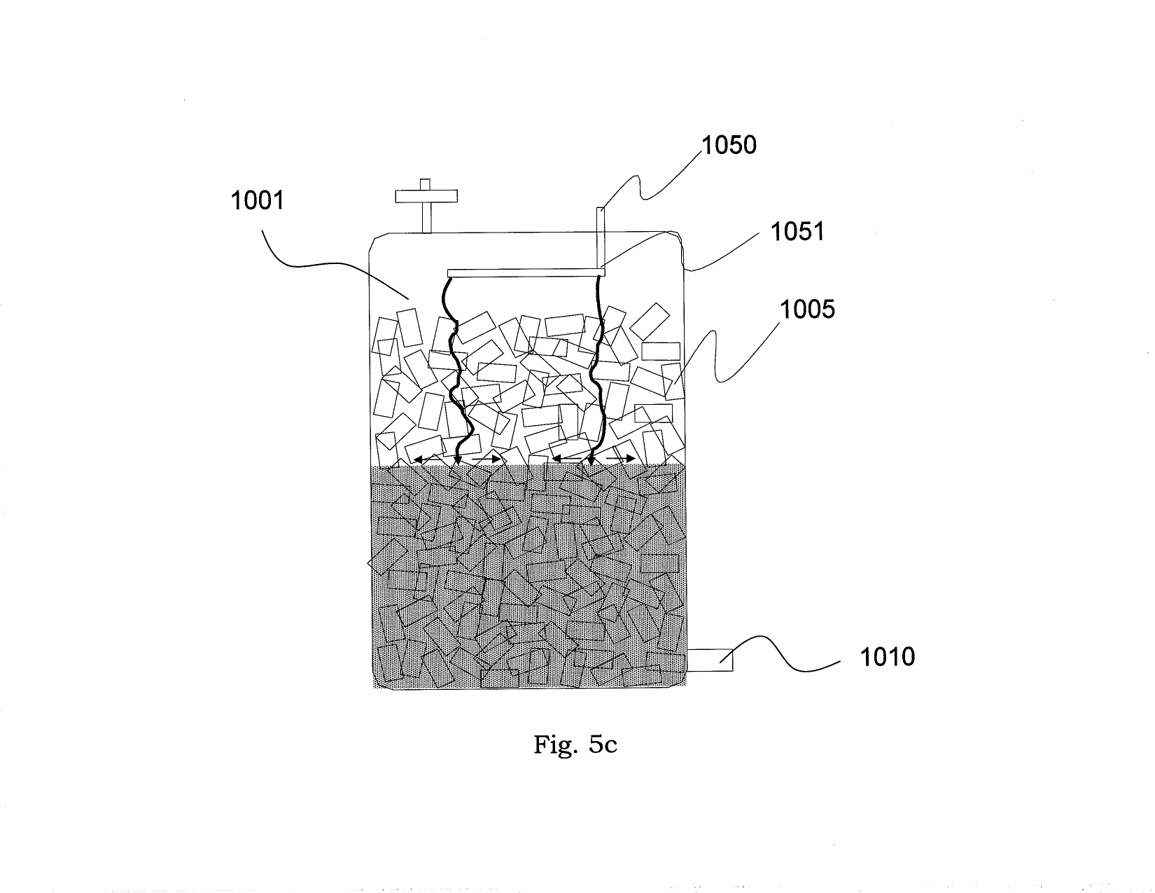

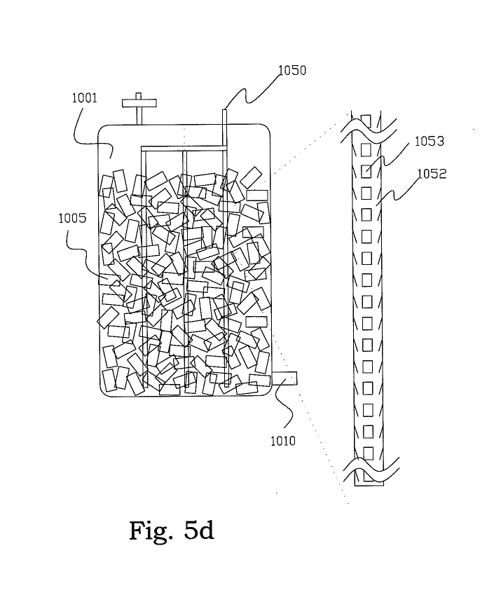

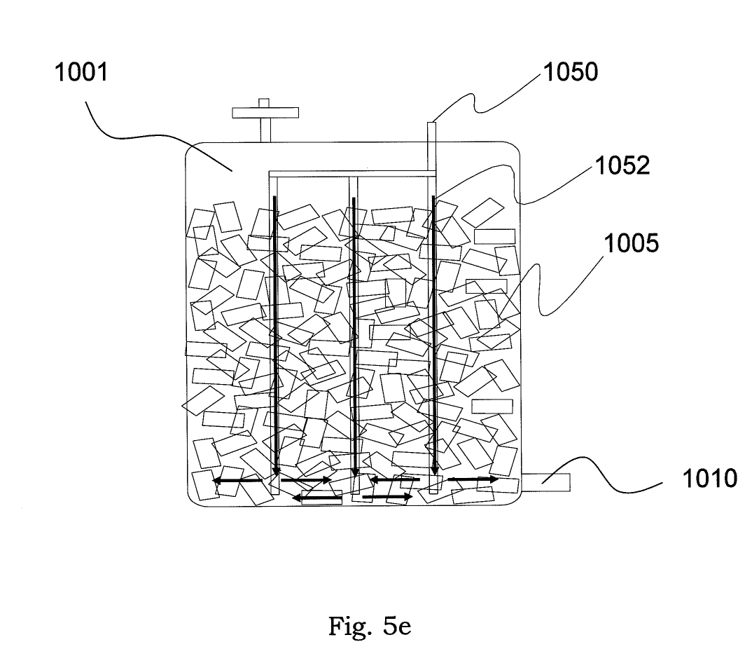

Referring to FIG. 4 the cell-cultivating apparatus of one of the preferred embodiment of invention is similar to FIG. 3 except the driving means for the culture medium flow between the matrix and mixing chamber is a pneumatic means, which is an air pump with solenoid valves and timer control 1034 set on a tube connected to air filter 1003 on the matrix vessel 1001 and the air filter 1003′ on the mixing vessel to control the flow of culture medium between the matrix vessel 1001 and the mixing vessel 1002 by pressure. The medium level in the matrix vessel 1001 is further controlled by a load cell 1035 which can be a level sensor as well. The mixing vessel 1002 contains a magnetic stir blade or a magnetic bar 1017 inside the chamber and is driving by a magnetic stirrer 1033 outside of the chamber 1002. Referring to FIGS. 5a and 5b, which are respectively a side-view and a top-view illustrating the inoculating device 1050, the inoculating device 1050 has a plurality of inoculating outlets 1051 for inoculation. Preferably, the inoculating device 1050 is of ring-shaped, and the inoculating outlets 1051 of the inoculating device 1050 are symmetric; however, it is not thus limited.

Referring to FIGS. 5a and 5c, in one preferred embodiment, the method for cell inoculation and cell-culturing in the present invention comprises the following steps: pre-sterilize the mixing vessel (not illustrated) and the matrix vessel 1001 which contains porous matrixes 1005, securing the mixing vessel on a platform with temperature control and also with mixing means that could homogenize the culture medium contained, aseptically fill the culture medium into the mixing vessel, securing the matrix vessel 1001 on another platform with temperature control, connect the matrix vessel 1001 to air and CO₂ gas with a controller, aseptically connect the matrix vessel to the mixing vessel, introducing cell-laden culture medium as inoculum medium to the matrix vessel 1001, preferably from the void space located on the top of the porous matrixes 1005 until the inoculum medium flows through the void space (mostly greater than 1 mm) formed among the porous matrixes 1005 and submerges the void space, intermittently move the culture medium up and down with short vertical distance, preferably less than or equal to the average height of the porous matrixes 1005, to distribute the cells and allow cells to attach on the matrixes 1005 evenly, after a period of time after cells are immobilized in the matrixes 1005, starting the driving means (not illustrated) to allow the culture medium flowing between two chambers intermittently and alternatively so that the porous matrixes 1005 can be submerged or exposed for any desirable time period in each cycle, whereby the necessary carbon dioxide and nutrients being transferred/mixed and nutrient concentration available to cells being controlled when the substrate is submerged and whereby oxygen is received through a thin medium film without directly contacting air when the substrate is exposed. As illustrated in FIG. 5c, the porous matrixes 1005 are then submerged with inoculum medium after inoculation.

Referring to FIGS. 5d and 5e, in another preferred embodiment of the present invention, the inoculating device 1050 has at least one guide tube 1052 having a plurality of holes 1053 and inserted into the porous matrixes 1005. The inoculating medium is then dispensed through the holes 1053 of the guide tubes 1052 to submerge the porous matrixes 1005. The inoculums in the inoculum medium would less be blocked by the porous matrixes 1005 and result in less vertical clogging.

The major difference between conventional inoculation method and the novel inoculation method is during inoculation, the matrix vessel is filled with culture medium in the conventional method, and a concentrated inoculums are introduced from top of the vessel filled with culture medium. While the novel method is started with a matrix vessel without being submerged with culture medium, and load with a well mixed inoculums with culture medium volume sufficient to submerge the porous matrixes; therefore, without being interfered by the channeling effect, a homogeneous inoculum solution is introduced into the matrix vessel. In addition, the inoculums in the homogeneous inoculum solution would distribute horizontally for decreased horizontal gradient. Furthermore, the inoculums in the inoculum solution flowing via the guide tubes configured within the porous matrixes would not be clogged by the depth filter formed by porous matrixes and thus achieve even more homogenous vertical distribution.

### Example 1

### Cell Distribution with the Novel Cell Inoculation Method

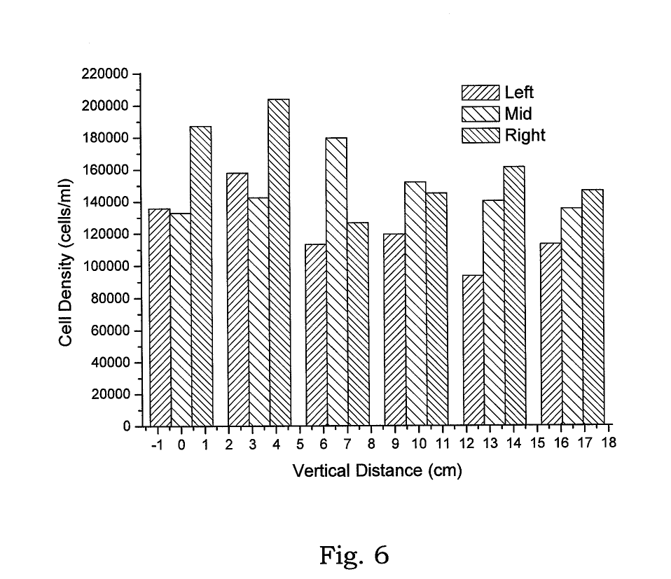

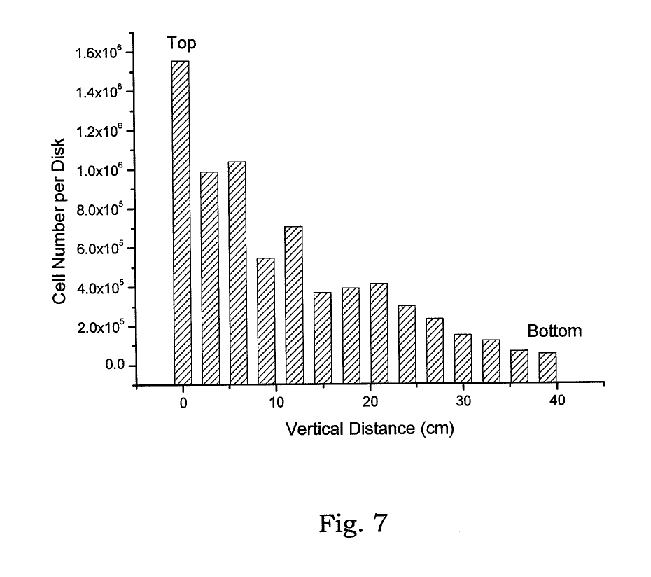

Prepare one cylinder with 54 cm high and 6 cm in diameter. Fill the cylinders with BioNOC II matrixes (products from CESCO Bioengineering Co., Ltd., www.cescobio.com.tw). Prepare cell culture medium 1.5 L containing well-mixed 1.1×10⁶ cells/ml. Introduce the cell laden culture medium into the cylinder from top-right by peristaltic pumping until the void space among the matrixes is filled with the culture medium. Place the cylinder into CO₂ incubator and allow sitting for 3 hours. After 3 hours, pick matrix samples from top of the cylinder every 9 cm vertical distance and every 3 cm horizontal distance. For comparison purpose, another experiment was executed with conventional inoculation method by introducing concentrated inoculums into a matrix vessel with 40 cm height and packed with BioNOC II carriers and pre-filled with culture medium. The medium was then started recirculated from top to bottom for 3 hours. After 3 hours, pick matrix samples from top of the cylinder every 3 cm vertical distance. FIG. 6 shows the result of cell distribution with the novel inoculation method. FIG. 7 shows the result of cell distribution with conventional inoculation method. The result indicates that there is no apparent gradient distribution along with the vertical distance in the cylinder. However, the conventional method has apparent cell distribution gradient along with the vertical distance in the vessel. It means the seeding protocol of the present invention can alleviate the gradient distribution in conventional inoculation method in packed-bed bioreactors.

### Example 2

### Cell Culture and Virus Production

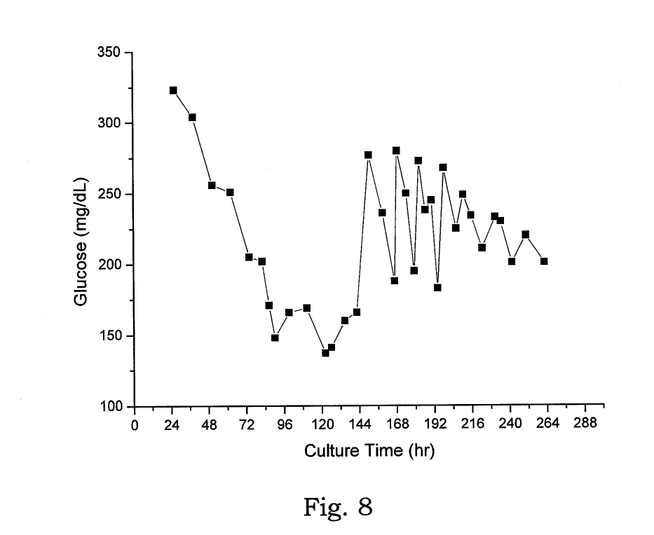

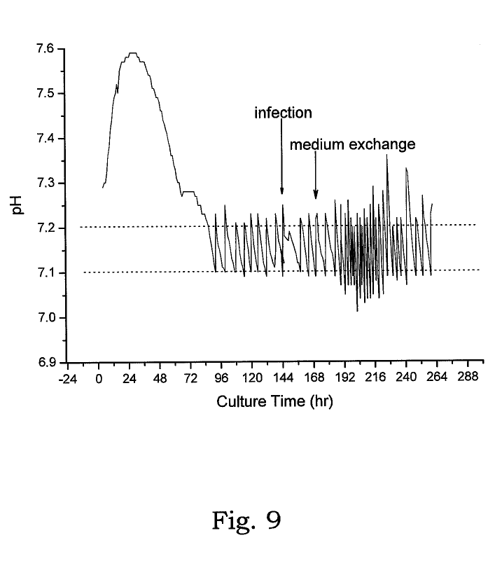

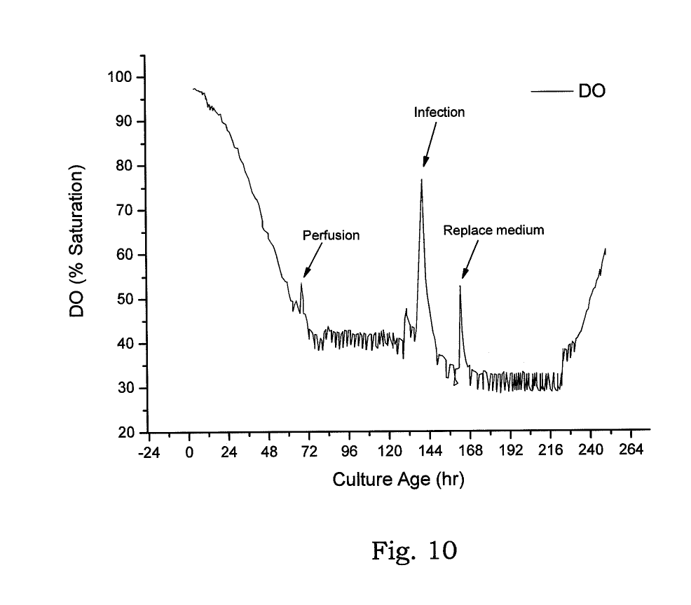

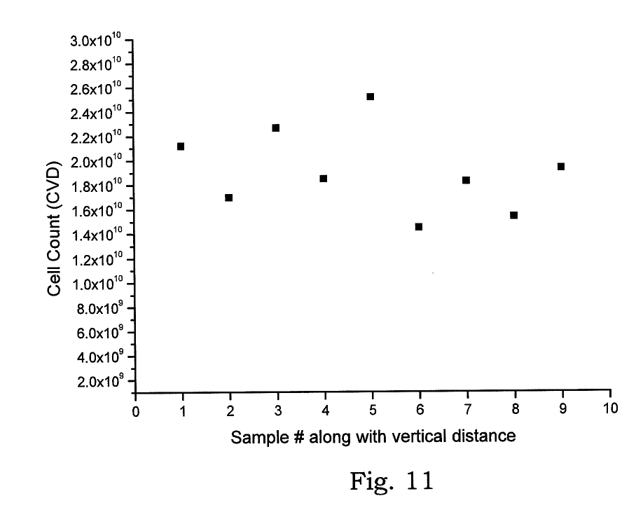

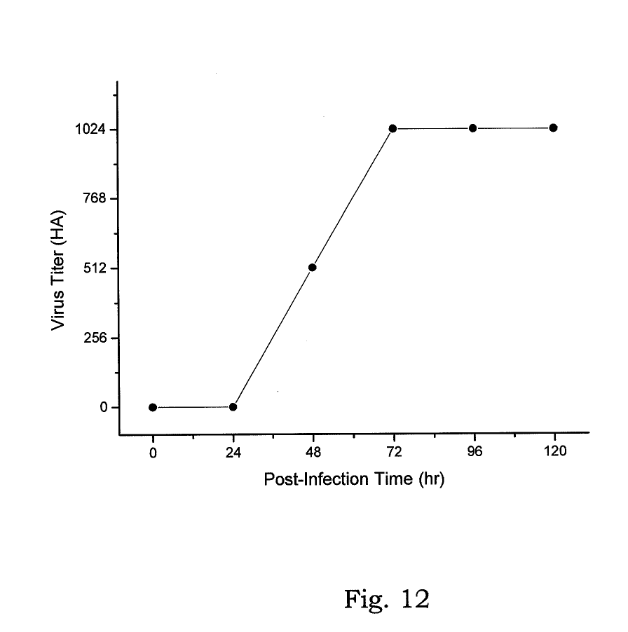

The culture device is constructed according to FIG. 3 except the mixing tank was constructed by a flexible bag in a shaker in stead of magnetic stirrer. Namely, the mixing vessel is a 50 L flexible medium bag placed in a thermostatic shaker with rotating rate and temperature control; the matrix vessel is a 10 L glass vessel packed with BioNOC II carriers. Two chambers are connected with a ½″ silicone tube and clamped to stop the medium flow between the two chambers. The medium flow is controlled by an air pump and a vacuum pump with timer control, and is connected to the matrix vessel with a silicone tube. There is a 0.22 um air filter between the pumps and the matrix vessel in order to prevent contamination. The 50 L flexible medium bag was filled with 40 L culture medium, namely DMEM/5% FBS. A glass vessel containing 7 L culture medium with 1×10⁶ cells/ml of MDCK cells was loaded into the 10 L glass vessel packed with BioNOC II carriers from top inlet until the vessel was filled with the culture medium. The clamp on the silicone tube was then opened and two chambers are connected. One liter of culture medium was drew from the mixing vessel to the matrix vessel and stayed for 30 seconds. Then one liter of culture medium was pushed back to the mixing vessel from the matrix vessel and stay for another 30 seconds. The circulation was continued for 4 hours until all cells are immobilized in the matrixes in the matrix vessel. After 4 hours, the culture medium in the matrix vessel was pushed to the mixing vessel completely and the matrixes were emerged from the culture medium and expose to the gaseous phase for oxygenation. Fresh and conditioned culture medium was fed and harvested from the feed tank and harvest tank. The feeding and harvest rate is according to the glucose consumption rate and control the minimum glucose concentration not lower than 1.0 g/L. The cycle was continued for six days until cell density reaches above 1×10⁷ cells/ml in the matrix vessel. Matrix samples were taken along with the vertical direction to examine the cell distribution. Cells were disrupted by crystal violet dye and citric acid, and nuclei were released from the matrixes. Nuclei count was done by hematocytometer. 10⁶H1N1 viruses were then loaded into the mixing vessel together with TPCK-treated trypsin with final concentration of 2 ug/ml. The culture was continued until cells were disrupted and viruses were released. Samples were taken for virus titer measurement every day. The result is shown in FIGS. 8˜12. FIG. 8 shows the glucose consumption with the cell culture device. The glucose concentration was controlled above 1.0 g/L by perfusion and feeding with concentrate. FIG. 9 shows the pH profile with the cell culture device. The pH was controlled between 7.1 to 7.2. FIG. 10 shows the dissolved oxygen profile with the cell culture device. Dissolved oxygen was controlled above 25% by introducing air or oxygen from the mixing vessel and the matrix vessel. FIG. 11 shows the cell distribution in the 10 L packed-bed vessel sampling before virus infection in the vertical direction from top to the bottom. There is no distribution gradient shown in the result. FIG. 12 shows the virus production profile with the cell culture device. The virus titer could reach 1024 HA/50 ul by 72 hours post-infection. With the present invention, the cell density could reach above 1×10¹¹ cells in one 10 L matrix vessel and H1N1 virus titer could reach 1024 HA/50 ul.

To sum up, the present invention provides a cell cultivating device, inoculation method and culture method that could eliminate the limitation of aeration or oxygenation during culture, can alleviate the gradient effect, and eliminate the channeling effect in conventional packed-bed bioreactors. The inoculation method provided by the present invention could enhance a homogenized cell distribution in large scale packed-bed bioreactor. Above all, the present invention provides a cell cultivating device, inoculation method and culture method that may be scaled up easily to any practical production scale because of its unique design feature on sufficient oxygen supply, eliminating gradient effect and channeling effect in conventional packed-bed cell culture device, and an improved inoculation method.

While the invention can be subject to various modifications and alternative forms, a specific example thereof has been shown in the drawings and is herein described in detail. It should be understood, however, that the invention is not to be limited to the particular form disclosed, but on the contrary, the invention is to cover all modifications, equivalents, and alternatives falling within the spirit and scope of the appended claims.

What is claimed is:

1. A scalable packed-bed cell culture device, comprising:
a matrix vessel comprising porous matrixes packed therein;
a mixing vessel comprising a mixing means configured for mixing a culture medium;
a communicating means connected between the matrix vessel and the mixing vessel;
a driving means configured for driving the culture medium to flow between the matrix vessel and the mixing vessel; and
a controlling means configured for controlling the culture medium to submerge the porous matrixes at high level, and to emerge the porous matrixes at low level.

2. The device as claimed in claim 1, wherein the driving means includes an air compressor, air pump or pressure/vacuum pump.

3. The device as claimed in claim 1, wherein the driving means is configured for vertically moving the mixing vessel or the matrix vessel so as to adjust the relative altitude between the matrix vessel and the mixing vessel.

4. The device as claimed in claim 1, wherein the mixing vessel further includes an oxygenation means configured for increasing the dissolved oxygen in the mixing vessel.

5. The device as claimed in claim 1, wherein the controlling means includes a liquid level sensor or a timer.

6. The device as claimed in claim 1, wherein the matrix vessel further comprises an inoculating device configured for introducing an inoculating medium into the porous matrixes.

7. The device as claimed in claim 6, wherein the inoculating device comprising at least one guide tube having a plurality of holes and inserted into the porous matrixes.

8. The device as claimed in claim 1, wherein the porous matrix includes woven carriers, non-woven carriers, plates, porous carriers made of ceramics, porous carriers made of polymer or tissue engineering scaffolds.

9. An inoculation method for a scalable packed-bed cell culture device, including:
providing a matrix vessel comprising porous matrixes packed therein, wherein a plurality of void space is formed among the porous matrixes; and
introducing an inoculum medium having an inoculum into the matrix vessel, wherein the inoculum medium flows through the void space and submerges the void space, whereby the inoculum is distributed onto the surface of the porous matrixes.

10. The inoculation method as claimed in claim 9 further comprising:
vertically oscillating the inoculum medium for a period of time.

11. The inoculation method as claimed in claim 9, wherein the inoculum medium is introduced via at least one of the void space located at the top of the porous matrixes.

12. The inoculation method as claimed in claim 9, wherein the porous matrix includes woven carriers, non-woven carriers, plates, porous carriers made of ceramics, porous carriers made of polymer or tissue engineering scaffolds.

13. The inoculation method as claimed in claim 9, wherein the inoculum includes eukaryotes, prokaryotes, animal cells or mammalian cells.

14. The inoculation method as claimed in claim 9, wherein the inoculum medium is introduced into the porous matrixes via at least one guide tube having a plurality of holes and inserted into the porous matrixes.

15. A cell culture method for a scalable packed-bed cell culture device, comprising:
providing the scalable packed-bed cell culture device comprising:
a matrix vessel comprising porous matrixes packed therein, wherein a plurality of void space is formed among the porous matrixes;
a mixing vessel comprising a mixing means configured for mixing a culture medium;
a communicating means connected between the matrix vessel and the mixing vessel;
a driving means configured for driving the culture medium to flow between the matrix vessel and the mixing vessel; and
a controlling means configured for controlling the culture medium to submerge the porous matrixes at high level, and to emerge the porous matrixes at low level;
introducing an inoculum medium having an inoculum into the matrix vessel, wherein the inoculum medium flows through the void space and submerges the void space, whereby the inoculum is distributed onto the surface of the porous matrixes; and
dual-directional flowing of the culture medium between the matrix vessel and the mixing vessel for emerging and submerging the porous matrixes.

16. The cell culture method as claimed in claim 15 further comprising
vertically oscillating the inoculum medium for a period of time.

17. The cell culture method as claimed in claim 15, wherein the inoculum medium is introduced via at least one of the voids located at the top of the porous matrixes.

18. The cell culture method as claimed in claim 15, wherein the porous matrix includes woven carriers, non-woven carriers, plates, porous carriers made of ceramics, porous carriers made of polymer or tissue engineering scaffolds.

19. The cell culture method as claimed in claim 15, wherein the inoculum includes eukaryotes, prokaryotes, animal cells and mammalian cells.

20. The cell culture method as claimed in claim 15, wherein the driving means includes an air compressor, air pump or pressure/vacuum pump.

21. The cell culture method as claimed in claim 15, wherein the driving means is configured for vertically moving the mixing vessel so as to adjust the relative altitude between the matrix vessel and the mixing vessel.

22. The cell culture method as claimed in claim 15, wherein the mixing vessel further includes an oxygenation means configured for increasing the dissolved oxygen in the mixing vessel.

23. The cell culture method as claimed in claim 15, wherein the controlling means includes a liquid level sensor or a timer.

24. The cell culture method as claimed in claim 15, wherein the inoculum medium is introduced into the porous matrixes via at least one guide tube having a plurality of holes and inserted into the porous matrixes.
